(19) United States
(12) Patent Application Publication
(10) Pub. No.: US 2013/0237444 A1
(43) Pub. Date: Sep. 12, 2013

(54) GBM MOLECULAR CONTEXTS ASSOCIATED WITH PATIENT SURVIVAL

(71) Applicants: Michael Berens, Phoenix, AZ (US); Jeff Kiefer, Phoenix, AZ (US); Seunghan Kim, Phoenix, AZ (US)

(72) Inventors: Michael Berens, Phoenix, AZ (US); Jeff Kiefer, Phoenix, AZ (US); Seunghan Kim, Phoenix, AZ (US)

(73) Assignee: Translational Genomics Research Institute, Phoenix, AZ (US)

(21) Appl. No.: 13/798,003

(22) Filed: Mar. 12, 2013

Related U.S. Application Data
(60) Provisional application No. 61/609,839, filed on Mar. 12, 2012.

(51) Int. Cl. C12Q 1/68

(52) U.S. Cl. CPC C12Q 1/6886
USPC 506/9; 506/16

(57) ABSTRACT

This disclosure provides a glioblastoma biomarker profile comprising glioblastoma associated genetic aberrations. The profile is indicative of a cellular and physiological characteristic of a sample from a subject. The disclosure further provides methods for profiling glioblastoma cellular and physiological characteristics including subtype, survival and drug response.

### FIELD OF THE INVENTION

This invention relates to a glioblastoma biomarker profile that comprises glioblastoma associated genetic aberrations indicating the tumor subtypes, drug response, and patient survival. Further, it relates to methods of determining cellular and physiological characteristics such as tumor subtype, drug response and patient survival of a glioblastoma sample using the biomarker profile. The glioblastoma biomarker profile as disclosed is useful for glioblastoma diagnosis, prognosis and theragnosis.

### BACKGROUND OF THE INVENTION

Glioblastoma, also commonly referred to in the art as malignant glioma or glioblastoma multiforme (GBM), is the most frequent form of primary brain cancer and has an average life expectancy from time of diagnosis of nine months to one year. Gene expression-based molecular classification has grouped GBM into proneural, neural, classical, and mesenchymal subtypes which are defined by genomic characteristics, survival length, patient age and treatment response. The highly lethal nature of this tumor partly originates from the invasive phenotype that affords the tumor cells the ability to infiltrate adjacent brain tissues. In terms of eradicating this invasive disease, it is considered incurable using treatment modalities presently available. As a result, genes that drive the invasive behavior of glioblastoma are important diagnostic and prognostic markers of glioblastoma and are also targets for new treatment methods.

### SUMMARY OF THE INVENTION

One aspect of the present invention provides a glioblastoma biomarker profile. The profile comprises glioblastoma associated genetic aberrations, wherein the profile is indicative of a cellular and physiological characteristic of a sample from a subject. The cellular and physiological characteristic that can be profiled with these markers is selected from the group consisting of glioblastoma subtype, survival, and drug-response. Generally, the glioblastoma subtypes include classical, neural, proneural or mesenchymal glioblastoma. The drug-response is sensitivity or resistance to drugs selected from the group consisting of temozolomide (TMZ), TMZ PARP inhibitor, BEZ235, and sorafenib. The genetic aberrations in the biomarker profile of the present invention are amplifications of EGFR, miR-548a, miR-31, miR-146a, and miR-219, and deletions of miRNA-181, PTEN, CDH8, CDH11 and NF1. For example, amplifications of EGFR and deletions of PTEN are present in all glioblastoma subtypes; deletions of CDH8, CDH11 or NF1 are present in mesenchymal glioblastoma, and amplifications of miR-219, miR-548a, miRNA-181, miR-31 or miR-146a are present in proneural glioblastoma. Further, in the biomarker profile, the amplifications of miRNA-181, miR-31 or miR-146a are indicative of a median survival longer than 2 years; whereas the amplification of miR-219 is indicative of a longer survival than without amplification in any glioblastoma subtypes. In addition, the deletions of CDH8, CDH11 or NF1 are indicative of sensitivity to TMZ, but resistance to sorafenib and BEZ235; whereas amplifications of miRNA-181, miR-31 or miR-146a are indicative of sensitivity to TMZ, TMZ PARP inhibitor, BEZ235, and sorafenib.

Another aspect of the invention provides a method for profiling glioblastoma cellular and physiological characteristic of a sample from a subject. The general method comprises receiving the sample; and detecting the presence of one or more glioblastoma associated genetic aberrations in a biomarker profile. The cellular and physiological characteristic that can be profiled using this general method is selected from the group consisting of glioblastoma subtype, survival, and drug-response. Generally, the glioblastoma subtype is classical, neural, proneural or mesenchymal glioblastoma. The drug-response is response to drugs selected from the group consisting of temozolomide (TMZ), TMZ PARP inhibitor, BEZ235, and sorafenib. The genetic aberrations in the biomarker profile for the general method are amplifications of EGFR, miR-548a, miR-31, miR-146a, and miR-219, and deletions of miRNA-181, PTEN, CDH8, CDH11 and NF1. When applying the method, the presence of deletions of CDH8, CDH11 or NF1 is associated with mesenchymal glioblastoma, and the presence of amplifications of miR-219, miR-548a, miRNA-181, miR-31 or miR-146a are associated with proneural glioblastoma. Therefore, the general method further comprises a step of grouping the sample into a glioblastoma subtype based on the presence of deletions of CDH8, CDH11 or NF1, or amplifications of miR-219, miR-548a, miRNA-181, miR-31 or miR-146a. When applying the general method, the detection of the presence of amplifications of miRNA-181, miR-31 or miR-146a indicates a median survival longer than 2 years; and the presence of amplification of miR-219 is indicative of a longer survival in any glioblastoma subtypes. Therefore, the general method may further comprise a step of including a sample in a group with a median survival rate longer than 1 year based on the presence of amplifications of miRNA-181, miR-31, miR-146a or miR-219. In addition, when applying the general method, the detection of the presence of deletions of CDH8, CDH11 or NF1 are indicative of sensitivity to TMZ, but resistance to sorafenib and BEZ235 of the subject; whereas the presence of amplifications of miRNA-181, miR-31 or miR-146a are indicative of sensitivity to TMZ, TMZ PARP inhibitor, BEZ235, and sorafenib of the subject. As such, the general method may further comprise a step of treating the subject with TMZ based on the presence of deletions of CDH8, CDH11 or NF1, or to any of TMZ, TMZ PARP inhibitor, BEZ235, and sorafenib based on the presence of amplifications of miRNA-181, miR-31 or miR-146a.

A technique chosen from quantitative real-time PCR, microarray, Western blotting, ELISA, and immunohistochemistry may be used in the step of detecting the presence of one or more glioblastoma associated genetic aberrations of the general method.

Other aspects and iterations of the invention are described in more detail below.

### DETAILED DESCRIPTION OF THE INVENTION

The present invention provides a GBM molecular profile comprising aberrations associated with survival. Specifically, this GBM molecular profile comprises EGFR, miR-548a, miR-31, miR-146a, miR-219, and miRNA-181, PTEN, CDH8, CDH11 and NF1. Some aberrations in the GBM molecular profile are enriched in particular GBM subtypes. Therefore, by determining the drug sensitivity of the GBM subtypes, the invention also provides a method of identifying candidates for treatment with different drug regimens such as, TMZ, TMZ PARP inhibitor, BEZ235, and sorafenib using this GBM molecular profile.

I. Aberrations in mRNA, miRNA Expression and Copy Number Variants in Cancer Cells

A cell maintains a specific state (such as “healthy” or “tumor”) by tightly regulating a set of molecules. When exposed to environmental changes, the cell adjusts its regulatory mechanisms and transitions to another state significantly different from the original state. Such transition alters the manner in which the system reacts to inputs, and thus, changes the cellular context. Therefore, cellular contexts could be understood as a number of biologically homogeneous states where a set of genes and other molecular components are tightly regulated. As such, biological observations are a mixture of different cellular contexts. For example, cancer patients have different responses to a treatment, and each response is defined by a context utilizing a different regulatory mechanism.

Each cellular context is defined as a set of genes, exhibiting consistent transcriptional behavior across a set of samples, drawn from a cellular process governed by tightly regulated mechanism(s) involving the set of genes. In the cellular context defining set of genes, one or more of them function as driver genes and the rest as driven genes (also called passenger). Each context defines regulatory relationships between the driver genes and the driven genes. A snippet of such a context, defined as driver-passenger relationships, is called context motif. A driver for a context motif can be a passenger in another context motif; hence context motifs are inter-connected. The regulation of genes within cellular context is called conditioning. Disruption of such regulation is called crosstalk. Crosstalk is defined as the probability that the gene is being regulated by external control in a given context. Context-Specific gene regulatory networks constructed from high throughput data are based on two consistency metrics, crosstalk and conditioning, that measure the specificity and sensitivity of the context. On a context level, a driver in one context could be a passenger by another driver in another context. When such relationships are added to the implicit driver-driven relationships, the structure representing the relationships between contexts starts to emerge.

A context-mining algorithm using a probabilistic framework (Ramesh et al. 2010 Clustering Context-Specific Gene Regulatory Networks, Pacific Symposium on Biocomputing 2010, Big Island, Hi., US) has been employed to learn contexts from gene expression data (also called mRNA data, or mRNA expression data), and to identify context specific gene regulatory networks, and context specific driver genes and passenger genes. In addition to gene expression data, miRNA and copy number variant (CNV/aCGH) can be utilized for context-mining. Different types of data can be further integrated for context analysis, with the objective to take into account gene regulatory interactions by different kinds of molecules and, consequently, to further refine molecular contexts. By integrating multi-omic data, different types of driver-passenger relationships were considered: miRNA→miRNA, miRNA→mRNA, and mRNA→mRNA, where specific state of a driver can determine the states of passengers.

In the present invention, molecular contexts of glioblastoma were identified from the TCGA-GBM gene expression data, or TCGA-GBM miRNA and mRNA integrated data (TCGA, The Cancer Genome Atlas), and verified using new TCGA-GBM gene expression data and xenograft GBM expression data. A molecular profile comprising an array of aberrations in mRNA and miRNA expression and copy number variants that are associated with GBM subtypes, survival, and drug responses is disclosed.

The aberrations in this GBM molecular profile include amplifications of EGFR, miR-548a, miR-31, miR-146a, miR-219, and miRNA-181, and deletions of PTEN (PTEN tumor suppressor; UniProtKB/Swiss-Prot No. P60484), CDH8 (encoding Cadherin-8; UniProtKB/Swiss-Prot No. P55286), CDH11 (encoding Cadherin-11; UniProtKB/Swiss-Prot No. P55287), NF1 (encoding Neurofibromin; UniProtKB/Swiss-Prot No. P21359). EGFR amplifications and PTEN deletions are present in all GBM subtypes: classic, neural, proneural, and mesenchymal. Deletions of CDH8, CDH11 and NF1 are associated with mesenchymal subtype. Amplifications of miR-219, miR-548a, miRNA-181, miR-31 and miR-146a are associated with proneural subtype. Specifically, amplifications of miRNA-181, miR-31 and miR-146a are indicative of GBM survival in >50% patients with >2.4 yr survival (median survival) in comparison to the rest of GBM patients with <1.0 yr median survival. Amplification of miR-219 in all subtypes is indicative of longer survival.

Further, classical GBM subtype is sensitive to TMZ type drugs but shows resistance to sorafenib. Mesenchymal GBM subtype is sensitive to TMZ type drugs, but is resistant to sorafenib and BEZ235. Neural GBM subtype is sensitive only to the TMZ type drugs. Proneural GBM subtype is sensitive to all regimens. Some of the aberrations in the disclosed GBM molecular profile further provide basis for theragnosis relating to drugs, which include but are not limited to temozolomide (TMZ), TMZ PARP inhibitor, BEZ235, and sorafenib. For example, deletions of CDH8, CDH11 or NF1, associated with mesenchymal GBM subtype, are indicative of sensitivity to TMZ type drugs, but resistance to sorafenib and BEZ235. Amplifications of miRNA-181, miR-31 or miR-146a, associated with proneural GBM subtype, are sensitive to all regimens.

II. Biomarkers and Therapeutic Targets

Among the various aspects of the present disclosure is the provision of a GBM molecular profile that comprises an array of aberrations, which can be used as biomarkers for diagnosis, prognosis and theragnosis. As use herein, the term “biomarker” refers to a gene and its gene products (i.e., RNA and protein) whose expression is indicative of a particular phenotype or cellular condition, or physiological characteristic. As one exemplary example, when a gene is up-regulated in a particular cellular or physiological state, the over-expression of these genes and their RNA or protein products may be used as biomarkers that indicate an association with the particular cellular or physiological characteristic.

Prediction of a cellular or physiological characteristic may be achieved by assessing the expression of one or more biomarkers, which may be genes or non-coding small RNAs (microRNA, or miRNA).

Genes or their alleles with disrupted expression may be used as diagnostic, prognostic or theranostic biomarkers. Different expression may be because of various gene alleles in different cellular context or gene copy number difference due to amplification or deletion. An allele includes any form of a particular nucleic acid that may be recognized as a form of the particular nucleic acid on account of its location, sequence, expression level, expression specificity, or any other characteristic that may identify it as being a form of the particular gene. Alleles include but need not be limited to forms of a gene that include point mutations, silent mutations, deletions, frameshift mutations, single nucleotide polymorphisms (SNPs), inversions, translocations, heterochromatic insertions, and differentially methylated sequences relative to a reference gene, whether alone or in combination. An allele of a gene may or may not produce a functional protein; may produce a protein with altered function, localization, stability, dimerization, or protein-protein interaction; may have over-expression, under-expression or no expression; or, may have altered temporal or spacial expression specificity.

An allele may be compared to another allele that may be termed a wild type form of an allele. In comparison to the wild type allele, a different allele may be called a mutation or a mutant. Mutants may also be interchangeably called variants. In some cases, the wild type allele is more common than the mutant. A genetic mutation or variance may be any detectable change in genetic material such as DNA, or a corresponding change in the RNA or protein product of that genetic material. In the example of gene mutation, the DNA sequence of a gene or any controlling elements surrounding the gene is altered. Controlling elements include promoter, enhancer, suppressor, or silencing elements capable of controlling a given gene. Other examples of mutations include alterations in the products of gene expression, such as RNA or protein, that result from corresponding mutations in the DNA.

Conserved variants encompass any mutation or other variant in which a given amino acid residue in a protein or enzyme has been changed without altering the overall conformation and function of the polypeptide, including, but not limited to, replacement of an amino acid with one having similar properties (for example, polarity, hydrogen bonding potential, acidic, basic, hydrophobic, aromatic, and the like). Amino acids with similar properties are well known in the art. For example, arginine, histidine and lysine are hydrophilic-basic amino acids and may be interchangeable. Similarly, isoleucine, a hydrophobic amino acid, may be replaced with leucine, methionine or valine. Depending on the location of the variance in the overall context of the protein, some substitutions may have little or no effect on the apparent molecular weight or isoelectric point of the protein or polypeptide.

Amino acids other than those indicated as conserved may differ in a protein or enzyme so that the percent protein or amino acid sequence similarity between any two proteins of similar function may vary and may be, for example, from about 70% to about 99% as determined according to an alignment scheme such as by the Cluster Method, wherein similarity is based on the MEGALIGN algorithm. The concept of a variant further encompasses a polypeptide or enzyme which has at least 60%, 75%, 85%, 90%, or 95% amino acid identity as determined by algorithms such as BLAST or FASTA and which has the same or substantially similar properties and/or activities as the native or parent protein or enzyme to which it is compared.

Another example of gene variant is a gain-of-function variant. Gain-of-function variants of polypeptides encompass any variant in which a change in one or more amino acid residues in a protein or enzyme improves the activity of the polypeptide. Examples of activities of a polypeptide that may be improved by a change resulting in a gain-of-function variant include but are not limited to enzymatic activity, binding affinity, phosphorylation or dephosphorylation efficiency, activation, deactivation, or any other activity or property of a protein that may be quantitatively measured by some method now known or yet to be disclosed.

The presence or absence of an allele may be detected through the use of any process known in the art, including using primers and probes designed according to a specific allele for PCR, sequencing, or hybridization analyses. Specifically, the gene biomarkers in the GBM molecular profile in this invention include amplifications of EGFR, deletions of PTEN, CDH8, CDH11, and NF1, wherein EGFR amplifications and PTEN deletions are associated with all subtypes of GBM, and deletions of CDH8, CDH11 and NF1 that are associated with mesenchymal GBM.

In addition to gene biomarkers, microRNAs (miRNAs) can be used as biomarkers for characterizing a cellular context as well. miRNAs are endogenous non-coding small RNAs, which negatively regulate gene expression either by binding to the 39 UTR leading to inhibition of translation or degradation of specific mRNA. Since miRNAs can act as oncogenes or tumor suppressor genes, they have been linked to a variety of cancers. Using miRNA expression biomarker predicting patient survival have been done in several cancers like lung cancer, lymphocytic leukemia; lung adenocarcinoma, breast and pancreas cancers. miRNA expression profiling can be carried out either by microarray or RT-PCR in cancer cells. Specifically, the miRNA biomarkers in the GBM molecular profile in this invention include amplification of miR-219, miR-548a, miR-31 and miR-146a, wherein amplifications of miR-219, miR-548a, miRNA-181, miR-31 and miR-146a are associated with proneural subtype, wherein amplifications of miRNA-181, miR-31 and miR-146a is indicative of GBM median survival >2.4 yr survival, wherein amplification of miR-219 is associated with all subtype and is indicative of longer survival in comparison to the non-carrier in any subtype.

GBM diagnosis, prognosis and theragnosis involve cellular or physiological characterization of the tumor cell, in which biomarkers associated with tumor subtype, tumor progression, patient survival and drug response are essential. One type of cellular or physiological characteristic of GBM is to group the tumor sample or patient into one of the four subtypes: classical, neural, proneural, and mesenchymal. Another type of cellular or physiological characteristic of GBM is the survival rate. Yet another type of cellular or physiological characteristic of GBM is the response of the tumor cell to a particular treatment. In one exemplary example, the treatment is temozolomide. The tumor cell may be responsive, non-responsive or becoming resistant after being responsive to the treatment for a time being. Assessing tumor response to a therapeutic drug includes the performing of any type of test, assay, and/or examination based on one or more biomarkers, from which the result, readout, or interpretation are correlated with an increased or decreased probability that an individual will be responsive or resistant to the therapy for a desired therapy outcome. Examples of therapy outcomes include, but need not be limited to survival, death, progression of existing disease, remission of existing disease, initiation of onset of a disease in an otherwise disease-free subject, or the continued lack of disease in a subject in which there has been a remission of disease. Assessing the risk of a disease outcome based on one or more biomarkers encompasses diagnosis in which the type of disease afflicting a subject is determined, specifically in this invention, a classical, neural, proneural or mesenchymal GBM. Assessing therapy outcome of a particular disease based on one or more biomarkers encompasses the concept of prognosis. A prognosis may be any assessment of the risk of disease outcome in an individual in which a particular type of disease has been diagnosed. Assessing the risk based on one or more biomarkers further encompasses theragnosis, that is, the prediction of therapeutic response in which a treatment regimen is chosen based on the assessment. Assessing the risk also encompasses a prediction of overall survival after diagnosis. In this invention, deletions of CDH8, CDH11 or NF1, associated with mesenchymal GBM subtype, are indicative of sensitivity to TMZ type drugs, but resistant to sorafenib and BEZ235. Amplifications of miRNA-181, miR-31 or miR-146a, associated with proneural GBM subtype, are sensitive to all regimens including temozolomide, TMZ PARP inhibitor, BEZ235, and sorafenib.

Some biomarkers may also be therapeutic targets for disease treatment. For example, inhibitors can be used to prevent the over-expression of a biomarker associated with a cellular or physiological characteristic to achieve therapeutic effects. In this sense, the over-expressed biomarker is a target for therapeutic purposes. Generally, a target may be any molecular structure produced by a cell, expressed inside the cell, accessible on the cell surface, or secreted by the cell. A target may be any protein, carbohydrate, fat, nucleic acid, catalytic site, or any combination of these, such as, an enzyme, glycoprotein, cell membrane, virus, cell, organ, organelle, or any uni- or multi-molecular structure or any other such structure now known or yet to be disclosed, whether alone or in combination. Specifically in this invention, a target may be represented by a nucleic acid sequence, the protein or peptide or the fragments thereof encoded by the nucleic acid sequence, such as EGFR, PTEN, CDH8, CDH11, or NF1, or microRNA such as miR-548a, miR-31, miR-146a, miR-219, and miRNA-181. Examples of such nucleic acid sequence include miRNA, tRNA, siRNA, mRNA, cDNA, or genomic DNA sequences. Further, a target may be represented by a nucleic acid sequence in a form with or without epigenetic modifications. In addition, a target may be represented by a nucleic acid sequence in a form with SNPs (single nucleiotide polymorphism), point mutations, silent mutations, deletions, insertions, frameshift mutations, translocations, and alternative splicing derivatives. Alternatively, a target may be represented by a protein, peptide, or the fragments thereof, with or without post-translational modifications.

III. Methods for Molecular Characterization of GBM Using Molecular Profile Comprising Biomarkers

The GBM molecular profile comprising biomarkers disclosed in the present invention provides a method to determine one or more characteristics of a sample, or a subject thereof, having or suspected to have GBM. These cellular or physiological characteristics include, but are not limited to, GBM subtype, survival and drug responses. Therefore, one aspect of this invention provides a method of grouping a sample, or a subject thereof, to a subtype of GBM, i.e., classical, neural, proneural, and mesenchymal GBM using the biomarker profile. Specifically, EGFR amplifications or PTEN deletions are present in all GBM subtypes, whereas deletions of CDH8, CDH11 or NF1 are associated with mesenchymal subtype. Amplifications of miR-219, miR-548a, miRNA-181, miR-31 or miR-146a are associated with proneural subtype.

Another aspect of this invention provides a method of predicting cancer survival of subjects having, or suspected to have, GBM, using the biomarker profile. Specifically, amplifications of miRNA-181, miR-31 and miR-146a are indicative of GBM survival >50% patients with >2.4 yr survival (median survival) in comparison to the rest of GBM patients with <1.0 yr median survival. Amplification of miR-219 in all subtypes is indicative of longer survival.

Yet another aspect of this invention provides a method of identifying subjects for treatment with a drug regime including, but not limited to, temozolomide (TMZ), TMZ PARP inhibitor, BEZ235, and sorafenib, and excluding subjects with drug-resistance from a regime treatment using biomarker profile. Specifically, deletions of CDH8, CDH11 or NF1, associated with mesenchymal GBM subtype, are indicative of sensitivity to TMZ type drugs, but resistant to sorafenib and BEZ235, and amplifications of miRNA-181, miR-31 or miR-146a, associated with proneural GBM subtype, are sensitive to all regimens.

The amplifications or deletions of a biomarker can be detected by methods determining its expression level. The expression of a biomarker in a test sample may be more or less than that of a predetermined level to predict the presence or absence of a cellular or physiological characteristic. The expression of the biomarker or target in the test subject may be 1,000,000×, 100,000×, 10,000×, 1000×, 100×, 10×, 5×, 2×, 1×, 0.5×, 0.1×, 0.01×, 0.001×, 0.0001×, 0.00001×, 0.000001×, or 0.0000001× of the predetermined level indicting the presence or absence of a cellular or physiological characteristic. The predetermined level of expression may be derived from a single control sample or a set of control samples.

1. Subject and Samples

One aspect of the invention provides assessing the expression of one or more biomarkers in a biological sample from a subject. A subject includes any human or non-human mammal, including primate, cow, horse, pig, sheep, goat, dog, cat, or rodent. A subject, including human patient, may be suspected of having GBM, may have been diagnosed with GBM, or may have a family history of GBM. Methods of identifying subjects suspected of having GBM include but are not limited to: physical examination, family medical history, subject medical history, biopsy, or a number of imaging technologies, such as, ultrasonography, computed tomography, magnetic resonance imaging, magnetic resonance spectroscopy, or positron emission tomography.

Examples of sources of samples include, but are not limited to, biopsy or other in vivo or ex vivo analysis of brain. In some aspects of the invention, the sample may be a body fluid sample, such as peripheral blood, serum, plasma, lymph fluid, ascites, serous fluid, pleural effusion, sputum, cerebrospinal fluid, amniotic fluid, lacrimal fluid, gastric fluid, pancreatic fluid, mucus or urine, from which free floating DNA, RNA, protein, peptide, or fragments thereof may be detected and compared to control samples. Samples include single cells, or tissue, in any condition including in vitro, ex vivo, in vivo, post-mortem, fresh, fixed, or frozen. Alternatively, a sample may be any cell source from which DNA, including genomic, somatic, and germline DNA, RNA, protein, peptide, or fragments thereof may be obtained.

The tumor cells in a sample may be obtained by any method now known in the art or yet to be disclosed, including for example, surgical resection, laser capture microdissection, isolation from blood or other fluids including lavage fluid, or any other method capable of obtaining and, if necessary, concentrating tumor cells. These tumor cells include any cells derived from a tumor, neoplasm, cancer, precancer, cell line, malignancy, or any other source of cells that have the potential to expand and grow to an unlimited degree. The GBM tumor cells may be derived from naturally occurring sources or may be artificially created cell lines. The GBM tumor cells may also be capable of invasion into other tissues and metastasis when placed into an animal host. The GBM tumor cells further encompass any malignant cells that have invaded other tissues and/or metastasized. One or more tumor cells of GBM in the context of an organism may also be called a cancer, neoplasm, growth, malignancy, or any other term used in the art to describe cells in a cancerous state.

2. Biomarker Expression Detection

The expression level of a biomarker can be determined, for example, by comparing mRNA or protein level in a test subject or sample to a control. In one embodiment of this invention, the comparison is between the test subject or sample and the non-GBM subject or sample, or a drug-responsive GBM subject or sample, as applicable. In one embodiment, the expression of the biomarker in a sample may be compared to a control level of expression predetermined to predict the presence or absence of a particular physiological characteristic, such as, subtype, survival, or drug-response. The predetermined control level of biomarker expression may be derived from a single control or a set of controls. Alternatively, a control may be a sample having a previously determined control level of expression of a specific biomarker. Comparison of the expression of the biomarker in the sample to a control level of expression results in a prediction that the sample exhibits or does not exhibit the cellular or physiological characteristic.

Expression of a biomarker may be assessed by any number of methods used to detect material derived from a nucleic acid template used currently in the art and yet to be developed. Examples of such methods include any biomarker nucleic acid detection method such as the following nonlimiting examples, microarray analysis, RNA in situ hybridization, RNase protection assay, Northern blot, reverse transcriptase PCR, quantitative PCR, quantitative reverse transcriptase PCR, quantitative real-time reverse transcriptase PCR, reverse transcriptase treatment followed by direct sequencing. Other examples include any method of assessing biomarker protein expression, such as, flow cytometry, immunohistochemistry, ELISA, Western blot, immunoaffinity chromatography, HPLC, mass spectrometry, protein microarray analysis, PAGE analysis, isoelectric focusing, 2-D gel electrophoresis, or any enzymatic assay.

Other methods used to assess biomarker expression include the use of natural or artificial ligands capable of specifically binding a biomarker or a target. Such ligands include antibodies, antibody complexes, conjugates, natural ligands, small molecules, nanoparticles, or any other molecular entity capable of specific binding to a target. The term “antibody” is used herein in the broadest sense and refers generally to a molecule that contains at least one antigen binding site that immunospecifically binds to a particular antigen target of interest. Antibody thus includes, but is not limited to, native antibodies and variants thereof, fragments of native antibodies and variants thereof, peptibodies and variants thereof, and antibody mimetics that mimic the structure and/or function of an antibody or a specified fragment or portion thereof, including single chain antibodies and fragments thereof. The term thus includes full length antibodies and/or their variants as well as immunologically active fragments thereof, thus encompassing, antibody fragments capable of binding to a biological molecule (such as an antigen or receptor) or portions thereof, including but not limited to, Fab, Fab′, F(ab′)2, facb, pFc′, Fd, Fv or scFv (See, e.g., CURRENT PROTOCOLS IN IMMUNOLOGY, (Colligan et al., eds., John Wiley & Sons, Inc., NY, 1994-2001).

Ligands may be associated with a label such as a radioactive isotope or chelate thereof, dye (fluorescent or nonfluorescent,) stain, enzyme, metal, or any other substance capable of aiding a machine or a human eye from differentiating a cell expressing a target from a cell not expressing a target. Additionally, expression may be assessed by monomeric or multimeric ligands associated with substances capable of killing the cell. Such substances include protein or small molecule toxins, cytokines, pro-apoptotic substances, pore forming substances, radioactive isotopes, or any other substance capable of killing a cell.

In addition, biomarker differential expression encompasses any detectable difference between the expression of a biomarker in one sample relative to the expression of the biomarker in another sample. Differential expression may be assessed by a detector, an instrument containing a detector, or by aided or unaided human eye. Examples include but are not limited to differential staining of cells in an IHC assay configured to detect a target, differential detection of bound RNA on a microarray to which a sequence capable of binding to the target is bound, differential results in measuring RT-PCR measured in ACt or alternatively in the number of PCR cycles necessary to reach a particular optical density at a wavelength at which a double stranded DNA binding dye (e.g., SYBR Green) incorporates, differential results in measuring label from a reporter probe used in a real-time RT-PCR reaction, differential detection of fluorescence on cells using a flow cytometer, differential intensities of bands in a Northern blot, differential intensities of bands in an RNase protection assay, differential cell death measured by apoptotic markers, differential cell death measured by shrinkage of a tumor, or any method that allows a detection of a difference in signal between one sample or set of samples and another sample or set of samples.

Techniques using microarrays may also be advantageously implemented to detect genetic abnormalities or assess gene expression. Gene expression may be that of the one or more biomarkers chosen from EGFR, PTEN, CDH8, CDH11, NF1, miR-548a, miR-31, miR-146a, miR-219, miRNA-181, or the expression of another set of genes upstream or downstream in a pathway of which the one or more biomarkers is a component or a regulator. In one embodiment, microarrays may be designed so that the same set of identical oligonucleotides is attached to at least two selected discrete regions of the array, so that one can easily compare a normal sample, contacted with one of said selected regions of the array, against a test sample, contacted with another of said selected regions. Examples of microarray techniques include those developed by Nanogen, Inc. (San Diego, Calif.) and those developed by Affymetrix (Santa Clara, Calif.). However, all types of microarrays, also called “gene chips” or “DNA chips”, may be advantageously implemented to detect genetic abnormalities or assess gene expression, e.g., adapted for the identification of mutations. Such microarrays are well known in the art.

In one embodiment of detecting the presence of a biomarker associated with a characteristic of a disease, a threshold value may be obtained by performing the one or more above mentioned assays on samples obtained from a population of patients having a certain disease condition (e.g., subtype, drug-response, survival) and from a second population of subjects that do not have the disease condition. In assessing disease outcome or the effect of treatment, a population of patients with a disease condition may be followed for a period of time. After the period of time expires, the population may be divided into two or more groups based on one or more parameters. For example, the population may be divided into a first group of patients whose disease progresses to a particular endpoint and a second group of patients whose disease does not progress to the particular endpoint. Examples of endpoints include disease recurrence, death, metastasis, chemo response or resistance, or other clinically meaningful indexes. Based on the observation of the parameters, a predetermined level of expression of a biomarker for each group may be selected to signify a particular physiological or cellular characteristic, including, for example, identifying or diagnosing a particular disease, assessing a risk of outcome or a prognostic risk, or assessing the risk that a particular treatment will or will not be effective. If expression of the biomarker in a test sample is more similar to the predetermined expression of the biomarker in one group relative to the other group, the sample may be assigned a risk of having the same outcome as the patient group to which it is more similar.

Additionally, a predetermined level of biomarker expression may be established by assessing the expression of a biomarker in a sample obtained first from one patient, assessing the expression of the biomarker in additional samples obtained later in time from the same patient, and comparing the expression of the biomarker from the samples later in time with the previous sample(s). This method may be used in the case of biomarkers that indicate, for example, progression or worsening of disease, lack of efficacy of a treatment regimen, remission of a disease, or efficacy of a treatment regimen.

In a preferred embodiment, predicting a test sample or subject's tumor subtype, survival or response to a drug therapy, is based on the detection of an altered expression of one or more biomarkers provided in a GBM molecular profile in the test subject in comparison to the expression level in a subject responsive to the therapy, and the GBM molecular profile, as disclosed herein comprises EGFR, PTEN, CDH8, CDH11, NF1, miR-548a, miR-31, miR-146a, miR-219, and miRNA-181.

### EXAMPLES

The following non-limiting examples are included to illustrate the invention.

### Example 1

### Integration of Multi-Omic Data Into Context Analysis

Context-Specific gene regulatory networks constructed from high throughput data are based on two consistency metrics, crosstalk and conditioning, that measure the specificity and sensitivity of the context. Using context-mining algorithm (Ramesh et al. 2000), molecular contexts of glioblastoma (GBM) were identified from the TCGA-GBM gene expression data (TCGA, The Cancer Genome Atlas). TCGA-GBM gene expression data are GBM expression data obtained from 301 TCGA tumor samples, which were further standardized against 10 non-tumor brain (NTB) samples, and quantized to over-expression, no-change and under-expression compared to NTB. TCGA-GBM data were further analyzed by integrating multi-omic data into context analysis, with the objective to take into account gene regulatory interactions by different kinds of molecules and, consequently, to further refine molecular contexts. Three different data types were utilized, which include miRNA, mRNA, and copy number variant (CNV/aCGH). The following three types of driver-passenger relationship were considered, where specific state of a driver can determine the states of passengers.

TABLE 1

| driver | | passenger |
|---|---|---|
| miRNA | [symbol] | miRNA |
| miRNA | [symbol] | gene (mRNA) |
| gene (mRNA) | [symbol] | gene (mRNA) |

Contexts were first identified with the miRNA and mRNA expression data sets. Once contexts were identified, copy number variants were analyzed to identify genomic copy number aberrations with statistical significance in each context. The objective was to enhance context-mining by integrating different data types with a goal to find the source of regulation.

Data processing: From the TCGA-GBM data, 292 patient samples with both of miRNA and mRNA expression data were used for context analysis. Data were standardized by converting the original values to z-score values, with using 10 NTB samples as a reference. After standardization, all z-score values were quantized to one of three discrete values—‘1’ for over-expression, ‘0’ for no-change, and ‘−1’ for under expression compared to the normal case, using the absolute z-score value 1.645 as a threshold (change of more than 1.645 standard deviation from the mean of normal case expressions). mRNA data was also processed in the same manner for the 301 samples for the TCGA-GBM data. For the copy number data, 265 samples with available aCGH data were segmented using the circular binary segmentation (CBS) method, as suggested by the GISTIC algorithm for copy number data analysis (Beroukhim et al. Proc. Natl. Acad. Sci. USA.2007 Dec 11;104(50):20007-12)

Identified contexts: From the multi-omic context-mining analysis using both the miRNA and mRNA expression data, 64 contexts were identified. After filtering contexts with more than 10 genes and 10 samples, 12 contexts were selected for further investigation, as shown in Table 2. From the result, several contexts (C25, C35, C37, C54, and C59) were found to be driven by miRNAs only. Two contexts (C16 and C54) were found being enriched with the proneural subtype of GBM, and both contexts were associated with significantly longer survival patterns based on Cox hazard ratio analysis. On the contrary, 4 out of 5 contexts enriched with the mesenchymal subtype indicated shorter survival. This result of proneural contexts with longer survival and mesenchymal contexts with shorter survival is consistent with the fact that the mesenchymal subtype has been considered to be a more malignant form of GBM.

TABLE 2
Selected contexts obtained from multi-omic context-mining analysis using miRNA and mRNA

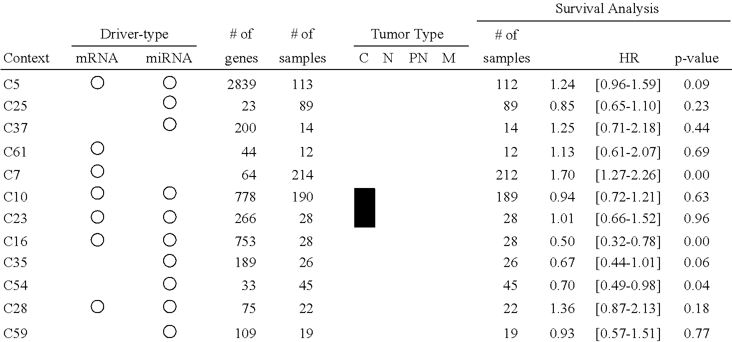

C = Classical; N = Neural; PN = Proneural; and M = mesenchymal; HR = Hazard ration

The two proneural enriched contexts, C16 and C54, showed different survival characteristics. The two proneural enriched contexts showed distinct survival characteristics. While Cox hazard ratio analysis (Cox, D. R. “Regression-Models and Life-Tables”. Journal of the Royal Statistical Society. B (Methodological) 34 (2): 187-220 (1972); Breslow, N. E.. “Analysis of Survival Data under the Proportional Hazards Model”. International Statistical Review / Revue Internationale de Statistique 43 (1): 45-57 (1975)) indicated both contexts with relatively longer survival, the hazard ratios of 0.50 (C16) and 0.70 (C54), and the median survival of those contexts revealed their differences. The patients in C16 have >2.4 years of median survival while the patients in C54 have about 1.4 yr of median survival which is still relatively longer than the rest of GBM patients with <1.0 yr median survival.

aCGH integration: Further integration of copy number variant data revealed potential driver of such difference between contexts C16 and C54. The copy number aberrations for each context, which were identified using the GISTIC analysis by measuring concordant events of amplification and deletion across each context (Beroukhim et al., 2007). The 12 identified contexts showed similar copy number changes as well as unique aberrations of their own. All contexts indicate consistent EGFR (Chr7) amplifications and PTEN (Chr10) deletions. However, C37 (enriched with Mesenchymal) shows deletions of CDH8 and CDH11 on Chr16, and C61 (Mesenchymal) indicates deletion of NF1 on Chr17. C35 (Proneural) shows amplification of miR-219 and miR-548a (Chr6). C16 (Proneural) indicates amplification of miR-31 and miR-146a (Chr9), where these two miRNAs were associated with GBM survival. Further, miRNA-181 was a driver for C16 and it was amplified in the same context. This suggests a new hypothesis if miRNA-181 plays a major role in representing the phenotype of C16 which include >50% patients with >2 yr survival. Another observation was the amplification of miR-219 for all contexts with longer survival, and this needs further investigation on its regulatory role on biological functions that can lead to better prognosis.

### Example 2

### Validation of Molecular Contexts of GBM

To evaluate the reproducibility of the 12 identified GBM contexts from TCGA-GBM mRNA expression data, each of the independent mRNA expression data set(s) from 182 new TCGA-GBM samples were mapped to the 12 identified contexts, and the characteristics of newly mapped sample were then compared to the context samples. The objective of mapping samples was to validate if the 182 samples mapped to each context identified in the context analysis based on 301 samples, have similar survival trends as the contexts.

First, the survival for 301 discovery samples were compared with the 182 validation samples using Cox regression analysis (Cox, D. R. & D. Oakes. 1984. Analysis of Survival Data. London: Chapman and Hall.), and the survival trends indicated that these 182 samples show similar survival and thus can be used for validation. New 182 samples were preprocessed employing the same technique used for the preparation of the 301 samples. 10 normal samples were used for the reference to convert GBM expression values to z-score values. All z-score values in GBM samples were also quantized to one of three discrete values: ‘1’ for over-expression, ‘0’ for no change, and ‘−1’ for under-expression compared to the normal case.

A couple of different mapping techniques to map the 182 additional samples to the 12 contexts were examined. In the first method, the centroids of each context were computed as the median of each gene across all samples. Two different distance metrics were used: correlation and normalized Mahalanobis distance. The distances were also normalized to account for the different number of genes within each context and each validation sample was assigned to the context with minimum distance. Secondly, using correlation as distance the 182 samples were compared to the individual context samples. Each validation sample is compared to a context by computing the correlation with all samples within the context. Three linkage methods were used in the comparison: single linkage, average linkage and complete linkage, to calculate the final distance of a sample to a context. In single linkage the distance between the context and sample was computed as the maximum correlation (or shortest distance) to the context samples. In average linkage the average of all distances was used as the distance and in complete linkage the minimum correlation (or maximum distance) of the sample to the context samples as distance was used.

A total of 44 samples among 182 samples had survival data available for analysis. The mapping based on correlation metric and average linkage showed the most consistent result, yielding only three contexts that show the survival trend opposite to that of context. The statistical analysis and the interpretation of the results can be further improved when more survival data are available and more samples are mapped to each context.

Further validation was provided using GBM tumor xenografts having drug response data as shown in Example 3.

### Example 3

### Validation of TCGA-GBM Gene Expression Data Contexts by Mapping GBM Tumor Xenograft Models

Model systems, such as xenografts, that capture the heterogeneity of GBM can be exploited to identify patterns within genomic profiles that are indicative of vulnerability to specific treatments. Those patterns can be further aligned with patient data for selection of therapy based on tumor profiling of patient GBM specimens. Such approach stipulates exploration of the differences and similarities between GBM xenografts and GBM patient samples. Note that xenografts have only mRNA data, the validation of TCGA-GBM contexts using xenografts therefore used only TCGA-GBM mRNA expression data, rather than miRNA and mRNA integrative data as in Example 1. Applying the similar context analyses using both miRNA and mRNA expression data of TCGA-GBM as in Example 1, context-specific gene interactions within patient mRNA expression data were investigated, and the result led to 12 sets of genes with corresponding subsets of samples with coherent expression profiles of those genes, termed TCGA-GBM gene expression contexts. Xenografts were mapped to individual gene expression contexts and preliminary survival analysis for different drug treatments was conducted. This work was focused on aligning xenografts to GBM patients with specific gene expression patterns so that such mappings can be beneficial in finding drug targets for diverse patient groups.

Forty GBM mouse xenografts were used. They were developed by the Ivy Genomics-Based Medicine (IGBM) project, with the evaluated response of up to 21 of those when treated with 4 different treatment protocols (temozolomide (TMZ), TMZ PARP inhibitor, BEZ235, and sorafenib). Because xenografts have only mRNA data, the gene expression data (not miRNA data) of 301 GBM patients and 10 non-tumor samples were used for GBM context-mining analysis in mapping xenograft models. These xenografts were also mapped to the four molecular subtypes: proneural, neural, classical, and mesenchymal.

The main step for mapping these xenografts include: identifying and eliminating confounding probes that are affected by the presence of murine RNA; subsequently using Kolmogorov-Smirnov (KS) test to yield genes that retain similar distributions between the expression data from xenograft and patient samples; mapping xenografts to molecular subtypes of GBM predetermined using TCGA-GBM patient samples; and characterizing potential drug responsiveness of GBM subtypes based on survival of xenografts treated with 4 drug regimens using Cox regression analysis.

Eliminating confounding probes: At first stage, 17,300 probes were removed from the controlled experiments with various amounts of contaminated murine RNAs. To remove probes that can accidentally capture the presence of mouse genes, 5,792 probes that correspond to mouse confounders based on sequence analysis, were removed. From the remaining gene expression probes, probes with similar expression distributions between the TCGA-GBM patient samples and the xenograft models were identified for the final analysis, using the KS test (with a p-value threshold of 0.001). The resulting 1,650 genes were used in this work to map the TCGA-GBM patient samples and the xenograft models. From the hierarchical clustering result of TCGA-GBM samples with the xenograft samples using the 1,650 genes, it was clear that xenograft samples are spread over the four different GBM subtypes, which supports the feasibility of mapping GBM patient samples to well studied xenograft models.

Mapping xenografts to GBM gene expression contexts and GBM subtypes: Xenograft models were mapped to the 12 TCGA-GBM gene expression contexts (C4, C5, C6, C8, C10, C13, C14, C16, C17, C21, C23, C25). For the features of mapping, the intersection of context genes and 1,650 non-confounding genes were used. Centroids were computed for each context using the continuous expression values across all samples. For each xenograft model, the distance between its gene expression values and the expressions of a context centroid is computed using the normalized Mahalanobis distance measure (Hazewinkel, Michiel, ed. (2001), “Mahalanobis distance”, Encyclopedia of Mathematics, Springer, ISBN 978-1-55608-010-4). Table 3 lists the result of mapping xenograft models to the 12 TCGA-GBM gene expression contexts. All four GBM subtypes are represented with xenograft models. However, mesenchymal (16-20 samples) and proneural (8 samples) subtypes seem more prevalent.

TABLE 3
Validation of TCGA-GBM gene expression contexts

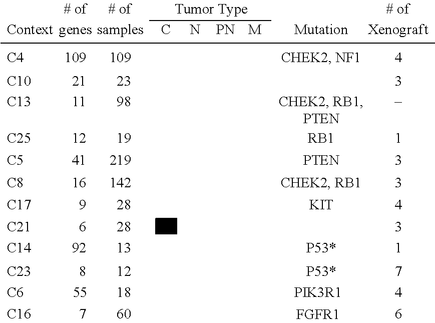

Table 3 shows the number of mapped xenografts to each TCGA-GBM gene expression context. The subtype column indicated the enrichment of GBM subtypes. Additionally, xenograft models can be directly mapped to the four subtypes by considering each subtype as a context. In this approach, a centroid of gene expressions was computed for each subtype, and the distance between a xenograft model and a subtype was computed in the same manner. The distance was computed using all 1,650 non-confounding genes, and the result was listed in Table 4. Two distance measures—normalized Mahalanobis distance and one minus correlation, were used in this process, and the result of mapping xenograft models to contexts were also listed for comparison. Again, all four GBM subtypes are represented by xenograft models, with mesenchymal and proneural being more prevalent than classical and neural.

TABLE 4
Result of direct mapping of xenograft models to each GBM subtype

| | Mapping based | | Mapping based | |
|---|---|---|---|---|
| | on subtypes | | on contexts | |
| | Mahalanobis | | Mahalanobis | |
| | distance | correlation | distance | correlation |
| Classical | 9 | 6 | 10 | 7 |
| Mesenchymal | 16 | 20 | 2 | 16 |
| Proneural | 13 | 12 | 7 | 8 |
| Neural, | — | — | 11 | 4 |
| Mesenchymal | | | | |
| Nueral | 1 | 1 | 4 | 1 |
| Unassigned | — | — | 5 | 3 |

Characterizing drug responsiveness for GBM subtypes using the mapped xenografts: Out of 40 xenograft models, 21 models were treated with 4 different treatments and their survival rates were compared against control groups. If a xenograft model with a treatment shows longer survival than its control group with statistical significance (by Cox regression analysis with a p-value threshold 0.05), the xenograft is declared as sensitive to the treatment. On the contrary, if it shows shorter survival, it is declared to be resistant to the treatment. Table 5 lists the frequency of treatment-sensitive and treatment-resistant xenograft model cases using 11 xenografts profiled for drug studies. In Table 5, NS denotes statistically Not Significant (by Cox regression analysis). The column with [symbol] indicates the cases with longer survival and the column with [symbol] indicates the cases with shorter survival.

This survival analysis of subtype-mapped xenografts treated with four drug regimens provided observations on subtype-specific drug responses. Classical-mapped xenograft models were sensitive to TMZ type drugs but showed resistance to sorafenib. Mesenchymal-mapped xenograft models also showed sensitivity to TMZ type drugs, but showed resistance to sorafenib and BEZ235. While Neural-mapped xenograft models were sensitive only to the TMZ type drugs, Proneural-mapped xenografts were sensitive to all regimens. This integration of xenograft models (expression data+survival data with drug treatment) provides information on sensitivity and/or resistance of GBM subtypes to specific drug, and could be a promising approach in finding drug targets for these patient groups.

TABLE 5
The frequency of significantly different survival samples within
each subtype and their sensitivity or resistance to the drug

| | | ABT888 + | | | | | | | | | | | |
|---|---|---|---|---|---|---|---|---|---|---|---|---|---|
| | | TMZ | | | TMZ | | | BEZ235 | | | Sorafenib | | |
| | Total | [symbol] | NS | [symbol] | [symbol] | NS | [symbol] | [symbol] | NS | [symbol] | [symbol] | NS | [symbol] |
| Classi- | 2 | 1 | 1 | 0 | 1 | 1 | 0 | 0 | 2 | 0 | 0 | 1 | 1 |
| cal | | | | | | | | | | | | | |
| Mesen- | 4 | 4 | 0 | 0 | 2 | 2 | 0 | 0 | 3 | 1 | 1 | 1 | 2 |
| chymal | | | | | | | | | | | | | |
| Neural | 3 | 1 | 2 | 0 | 1 | 2 | 0 | 0 | 3 | 0 | 0 | 3 | 0 |
| Pro- | 2 | 2 | 0 | 0 | 1 | 1 | 0 | 1 | 1 | 0 | 1 | 1 | 0 |
| neural | | | | | | | | | | | | | |

What is claimed is:

1. A glioblastoma biomarker profile, comprises glioblastoma associated genetic aberrations, wherein the profile is indicative of a cellular and physiological characteristic of a sample from a subject.

2. The biomarker profile of claim 1, wherein the cellular and physiological characteristic is selected from the group consisting of glioblastoma subtype, survival, and drug-response.

3. The biomarker profile of claim 2, wherein the glioblastoma subtype is classical, neural, proneural or mesenchymal glioblastoma.

4. The biomarker profile of claim 2, wherein the drug-response is determined by the response to drugs selected from the group consisting of temozolomide (TMZ), TMZ PARP inhibitor, BEZ235, and sorafenib.

5. The biomarker profile of claim 1, wherein the sample comprises a glioblastoma tumor cell.

6. The biomarker profile of claim 1, where in the subject is a mammal.

7. The biomarker profile of claim 6, wherein the mammal is a human.

8. The biomarker profile of claim 1, wherein the genetic aberrations are selected from the group consisting of amplifications of EGFR, miR-548a, miR-31, miR-146a, and miR-219, and deletions of miRNA-181, PTEN, CDH8, CDH11 and NFL

9. The biomarker profile of claim 8, wherein amplifications of EGFR and deletions of PTEN are present in all glioblastoma subtypes; wherein deletions of CDH8, CDH11 or NF1 are present in mesenchymal glioblastoma, and wherein amplifications of miR-219, miR-548a, miRNA-181, miR-31 or miR-146a are present in proneural glioblastoma.

10. The biomarker profile of claim 8, wherein amplifications of miRNA-181, miR-31 or miR-146a are indicative of a median survival longer than 2 years; and wherein amplification of miR-219 is indicative of a longer survival than without amplification in any glioblastoma subtypes.

11. The biomarker profile of claim 8, wherein deletions selected from the group consisting of CDH8, CDH11 or NF1 are indicative of sensitivity to TMZ, but resistance to sorafenib and BEZ235; whereas amplifications selected from the group consisting of miRNA-181, miR-31 or miR-146a are indicative of sensitivity to TMZ, TMZ PARP inhibitor, BEZ235, and sorafenib.

12. A method for profiling glioblastoma cellular and physiological characteristic of a sample from a subject, comprising
a. receiving the sample; and
b. detecting the presence of one or more glioblastoma associated genetic aberrations in a biomarker profile;
c. profiling glioblastoma cellular and physiological characteristics based on the results of said detecting step.

13. The method of claim 12, wherein the cellular and physiological characteristic is selected from the group consisting of glioblastoma subtype, survival, and drug-response.

14. The method of claim 13, wherein the glioblastoma subtype is selected from the group consisting of classical, neural, proneural or mesenchymal glioblastoma.

15. The method of claim 13, wherein the drug-response is response to drugs selected from the group consisting of temozolomide (TMZ), TMZ PARP inhibitor, BEZ235, and sorafenib.

16. The method of claim 12, wherein the sample comprises a glioblastoma tumor cell.

17. The method of claim 12, where in the subject is a mammal.

18. The method of claim 12, wherein the mammal is a human.

19. The method of claim 12, wherein the genetic aberrations are selected from the group consisting of amplifications of EGFR, miR-548a, miR-31, miR-146a, and miR-219, and deletions of miRNA-181, PTEN, CDH8, CDH11 and NF1.

20. The method of claim 19, wherein the presence of deletions selected from the group consisting of CDH8, CDH11 or NF1 is associated with mesenchymal glioblastoma, and wherein the presence of amplifications selected from the group consisting of miR-219, miR-548a, miRNA-181, miR-31 or miR-146a are associated with proneural glioblastoma.

21. The method of claim 20, further comprising a step of grouping the sample into a glioblastoma subtype based on the presence of deletions selected from the group consisting of CDH8, CDH11 or NF1, or amplifications of miR-219, miR-548a, miRNA-181, miR-31 or miR-146a.

22. The method of claim 19, wherein the presence of amplifications selected from the group consisting of miRNA-181, miR-31 or miR-146a are indicative of a median survival longer than 2 years; and wherein the presence of amplification of miR-219 is indicative of a longer survival in any glioblastoma subtypes.

23. The method of claim 22, further comprising a step of including a sample in a group with a median survival rate longer than 1 year based on the presence of amplifications selected from the group consisting of miRNA-181, miR-31, miR-146a or miR-219.

24. The method of claim 19, wherein the presence of deletions selected from the group consisting of CDH8, CDH11 or NF1 are indicative of sensitivity to TMZ, but resistance to sorafenib and BEZ235 of the subject; wherein the presence of amplifications selected from the group consisting of miRNA-181, miR-31 or miR-146a are indicative of sensitivity of the subject to TMZ, TMZ PARP inhibitor, BEZ235, and sorafenib of the subject.

25. The method of claim 24, further comprising a step of treating the subject to a TMZ based on the presence of deletions selected from the group consisting of CDH8, CDH11 or NF1, or to any of TMZ, TMZ PARP inhibitor, BEZ235, and sorafenib based on the presence of amplifications selected from the group consisting of miRNA-181, miR-31 or miR- 146a.

26. The method of claim 12, wherein the presence of one or more glioblastoma associated genetic aberrations is detected by a technique selected from the group consisting quantitative real-time PCR, microarray, Western blotting, ELISA, and immunohistochemistry.
